(19) United States
(12) Patent Application Publication
(10) Pub. No.: US 2020/0190296 A1
(43) Pub. Date: Jun. 18, 2020

(54) ELASTOMERIC FILM-FORMING COMPOSITIONS AND ASSOCIATED ARTICLES AND METHODS

(71) Applicant: SKINPROTECT CORPORATION SDN BHD, Klang, Selangor (MY)

(72) Inventors: Khon Pu Foo, Klang, Selangor (MY); Kumaresan Prabhakaran, Klang, Selangor (MY)

(73) Assignee: SKINPROTECT CORPORATION SDN BHD, Klang, Selangor (MY)

(21) Appl. No.: 16/474,758

(22) PCT Filed: Dec. 22, 2017
(86) PCT No.: PCT/AU2017/051450

§ 371 Date: Jun. 28, 2019

(30) Foreign Application Priority Data
Dec. 30, 2016 (AU) 2016-905396

(51) Int. Cl. C08L 13/00; A61L 31/04; C08J 5/02; A61B 42/10; B32B 25/16

(52) U.S. Cl. CPC C08L 13/00; A61L 31/049; C08J 5/02; A61B 42/10; B32B 2250/248; C08J 2313/00; C08J 2411/00; B32B 2437/02; B32B 25/16

(57) ABSTRACT

The invention relates to an elastomeric film-forming composition (a) a carboxylated butadiene-based elastomer, (b) polychlorobutadiene in an amount of less than 30% by weight of the polymer content of the composition, and (c) one or more cross-linking agents. The invention also relates to dipped articles, gloves, methods of manufacture and uses involving the composition.

### FIELD

The present invention relates to elastomeric film-forming compositions for use in manufacturing dipped articles, such as gloves, and articles made from the elastomeric film-forming compositions and methods for forming articles from the compositions.

### BACKGROUND OF THE INVENTION

Articles such as gloves that are made from natural (polyisoprene) rubber have favorable feel and comfort properties. However, natural (polyisoprene) rubber is associated with potential allergen (which causes Type I allergy). In view of this allergenic property, natural (polyisoprene) rubber is generally not suitable for use in the manufacture of articles such as rubber gloves due to the adverse effect of natural (polyisoprene) rubber on the wearer.

Other compositions that can be used to form gloves and other like articles are based on synthetic materials such as nitrile rubber, polyisoprene, styrene butadiene rubber, butyl rubber and vinyl polymers. Over the past few years the volume of glove production using synthetic materials has increased substantially. While such gloves are available, there is still the opportunity to further improve the gloves and to develop new variations having beneficial properties.

Compositions based on synthetic materials such as nitrile rubber have the potential for application in articles other than gloves. For example, dipped articles may be configured for use in medical applications such as surgical gloves, examination gloves, catheters, tubing, protective covering, balloons for catheters, condoms and like, or for use in non-medical applications, such as industrial gloves, laboratory gloves, household gloves, gardening gloves, electrical gloves, irradiation gloves, finger cots, weather balloons, clean room gloves for electronic industries, gloves for food contact and food processing and biotechnical application and like.

There is a need for new forms of compositions for producing such products, for the production of alternative or improved dipped articles, and associated methods of manufacturing the articles.

### SUMMARY

According to the present application, there is provided an elastomeric film-forming composition comprising:

(a) a carboxylated butadiene-based elastomer,

(b) polychlorobutadiene in an amount of less than 30% by weight of the polymer content of the composition, and

(c) one or more cross-linking agents.

The composition of the invention can be used to prepare thin elastomeric film layers, which may be created in the shape of an article such as a glove or otherwise. This composition produces products having very good properties in terms of elasticity, strength, durability and the absence of defects like pin holes or weak spots.

Component (b), the polychlorobutadiene, is a non-carboxylated polychlorobutadiene, but component (a) is carboxylated.

In one embodiment, the elastomeric film-forming composition of the invention can be used to form thin layers of elastomeric film. In another embodiment, the elastomeric film-forming composition of the invention can be used to prepare dipped articles, such as gloves, which may have improved properties such as improved feel, improved softness or increased elasticity.

In another embodiment, there is provided an elastomeric article comprising at least one layer of a cured composition comprising:

(a) a carboxylated butadiene-based elastomer,

(b) polychlorobutadiene in an amount of less than 30% by weight of the polymer content of the composition, and

(c) one or more cross-linking agents.

The elastomeric film may be in the form of a dipped article, where a former in the shape of an article is dipped into the elastomeric film-forming composition and the composition is cured on the former.

In another embodiment, there is provided a dipped article made from an elastomeric film comprising at least one layer of a cured composition comprising:

(a) a carboxylated butadiene-based elastomer,

(b) polychlorobutadiene in an amount of less than 30% by weight of the polymer content of the composition, and

(c) one or more cross-linking agents.

In another embodiment, there is provided a glove comprising at least one layer of elastomeric film comprising:

(a) a carboxylated butadiene-based elastomer,

(b) polychlorobutadiene in an amount of less than 30% by weight of the polymer content of the composition, and

(c) one or more cross-linking agents.

The elastomeric film, article or gloves may be made from an elastomeric film-forming composition according to any of the embodiments of the composition described herein.

The present inventors have identified that the combination of a carboxylated butadiene-based elastomer with less than 30% by weight of non-carboxylated polychlorobutadiene (the amount being based on the total polymer content of the composition), can be used to prepare dipped articles having beneficial properties. The dipped articles prepared from the elastomeric film-forming composition of the invention retain the favourable feel and comfort that is closer to natural rubber film yet is free of proteins and other potential allergens (causing Type I allergy) associated with natural rubber. Where the dipped article is a glove, the products are easily donnable without any visible powder anti tack material. The thickness of the layer of film of the glove or other article can also be very thin without compromising the elasticity, strength, durability or other characteristics such as feel, comfort, softness or the absence of defects, which allows the film to be used in specific applications such as medical examination gloves and surgical gloves, where it is important that the film does not prevent the wearer from having good tactile perception. Further, even though the applicant's prior work had indicated that, when using chlorobutadiene, this must be carboxylated, the applicant has now surprisingly found that it is possible to combine non-carboxylated polychlorobutadiene with carboxylated butadiene-based elastomers, provided that the amount of polychlorobutadiene is less than 30% (less than 30 phr), and to achieve excellent film properties. This avoids the need to perform any carboxylation on the polychlorobutadiene polymer previously thought to be necessary. The properties of the product containing chlorobutadiene are also superior to elastomers based on butadiene-based elastomers alone, or carboxylated butadiene-based elastomers alone. Gloves or other articles prepared from the elastomeric film-forming composition of the present invention can be made from very thin layers of elastomeric film and using a minimal amount of polymeric material while still maintaining industry requirements for the specific applications such as elasticity, strength, durability and the absence of defects like pin holes or weak spots. The use of less polymeric material also means that the product can be produced at a lower cost.

In some embodiments, the dipped articles prepared from the elastomeric film-forming composition of the invention have a lower modulus at 300%, a lower modulus at 500% and/or a higher elongation to break when compared to other elastomeric films used to form dipped articles or gloves. In some embodiments, the dipped articles prepared from the elastomeric film-forming composition of the invention have a tensile strength of greater than or equal to about 2000 psi, a modulus at 300% of about 100 to 2000 psi, a stress at 500% of about 200 to 3000 psi, and/or an elongation to break of about 400 to 1500%. For example, the elastomeric film prepared from the composition of the present invention has a tensile strength of at least about 2000 psi, a modulus at 300% of less than about 650 psi, a stress at 500% no greater than about 1500 psi, and/or an elongation to break of greater than 550%.

In a further embodiment, there is provided a method of manufacturing an elastomeric film comprising the steps of: (i) dipping a former into a composition as described above to produce a layer of elastomeric film-forming composition on the former, and (ii) drying and/or curing the elastomeric film-forming composition.

In one embodiment, the method will further comprise, prior to step (i), the steps of: (a) dipping the former into a coagulant, followed by (b) drying or partially drying the coagulant-dipped former.

In another embodiment, the method will further comprise, following step (ii), the steps of:

(iii) dipping the former into a composition as described above to produce a further layer of elastomeric film-forming composition on the former,

(iv) optionally repeating the drying step (ii) and the further dipping step (iii), and

(v) drying and curing the layered elastomeric film.

In some embodiments, the drying step and the dipping step are repeated to produce a film having from 2 to 15 layers. For example, a method for producing a film having two layers will require that the drying step and the further dipping step are repeated at least once.

In a further embodiment, there is provided a multiple-coating method of manufacturing a layered elastomeric film comprising the steps of:
(i) dipping a former into a composition as described above to produce a layer of elastomeric film-forming composition on the former,
(ii) drying or partially drying the elastomeric film-forming composition,
(iii) dipping the former into a composition as described above to produce a further layer of elastomeric film-forming composition on the former,
(iv) optionally repeating the drying step (ii) and the further dipping step (iii), and
(v) drying and curing the layered elastomeric film.

In another embodiment, there is provided an elastomeric film produced by the method as described above. The elastomeric film produced by the method as described above may involve the elastomeric film-forming composition according to any of the embodiments of the composition described herein.

In a further embodiment, there is provided the use of an elastomeric film-forming composition comprising:

(a) a carboxylated butadiene-based elastomer,

(b) polychlorobutadiene in an amount of less than 30% by weight of the polymer content of the composition, and

(c) one or more cross-linking agents, in the manufacture of a glove.

In a still further embodiment, there is provided an elastomeric film-forming composition comprising:

(a) a carboxylated butadiene-based elastomer,

(b) polychlorobutadiene in an amount of less than 30% by weight of the polymer content of the composition, and

(c) a covalent cross-linking agent (such as a sulphur-containing covalent cross-linking agent) and an ionic cross-linking agent,

wherein the elastomeric film-forming composition has a total solids content of 5% to 40% by weight of the composition, and all of the polychlorobutadiene component is non-carboxylated polychlorobutadiene.

In a still further embodiment, there is provided an unsupported elastomeric article comprising at least one layer of a cured composition comprising:

(a) a carboxylated butadiene-based elastomer,

(b) polychlorobutadiene in an amount of less than 30% by weight of the polymer content of the composition, and

(c) a covalent cross-linking agent (such as a sulphur-containing covalent cross-linking agent) and an ionic cross-linking agent,

wherein the elastomeric article has a thickness of 0.01-0.10 mm, and all of component (b) is non-carboxylated polychlorobutadiene.

In these embodiments, the ionic cross-linking agent may be zinc oxide in an amount of less than 2 phr.

Additional details concerning the dipped articles, their properties and their manufacture are described in further detail below.

### BRIEF DESCRIPTION OF THE DRAWINGS

Preferred embodiments of the present invention will now be further described and illustrated, by way of example only, with reference to the accompanying drawings.

FIG. 1 is a graph showing the elongation (%) results obtained for the elastomeric films obtained from the compositions of Examples 1 to 5. The X axis refers to the example number (Examples 1-5 in that order, with an increasing amount of polychloroprene from 5% to 27%), and the Y axis refers to the % elongation at break. Note that the lower line refers to the results following accelerated ageing (AA) and the upper line plots the results for unaged films (or before ageing—BA).

FIG. 2 is a graph showing the tensile strength results obtained for the same elastomeric films of Examples 1 to 5. The X axis refers to the example number, and the Y-axis refers to the tensile strength in MPa. Note that the lower line refers to the results following unaged films (or before ageing—BA) and the upper line plots the results for accelerated ageing (AA).

FIG. 3 is a graph showing the modulus at 500% results for the same elastomeric films of Examples 1 to 5. The X axis refers to the example number, and the Y axis refers to the modulus in MPa. Note that the lower line refers to the results following unaged films (or before ageing—BA) and the upper line plots the results for accelerated ageing (AA).

FIG. 4 is a graph showing the elongation (%) results obtained for the elastomeric films obtained from the compositions of Examples 6 to 9 and 5. The X axis refers to the percentage amount of polychloroprene (which increases from 5% to 27%, and the Y axis refers to the % elongation at break. Note that the lower line refers to the results following accelerated ageing (AA) and the upper line plots the results for unaged films (or before ageing—BA).

FIG. 5 is a graph showing the tensile strength results obtained for the same elastomeric films of Examples 6 to 9 and 5. The X axis refers to the percentage amount of polychloroprene, and the Y-axis refers to the tensile strength in MPa. Note that the lower line refers to the results following unaged films (or before ageing—BA) and the upper line plots the results for accelerated ageing (AA).

FIG. 6 is a graph showing the modulus at 500% results for the same elastomeric films of Examples 6 to 9 and 5. The X axis refers to the percentage amount of polychloroprene, and the Y axis refers to the modulus in MPa. Note that the lower line refers to the results following unaged films (or before ageing—BA) and the upper line plots the results for accelerated ageing (AA).

FIG. 7A is a schematic diagram showing an ASTM D412 Type C sample cutting die. FIG. 7B is a schematic diagram showing an ASTM D412 Type D sample cutting die. The ASTM D412 Type C and ASTM D412 Type D sample cutting dies may be used to prepare test specimens for measuring tensile strength, stress at 300% and 500% modulus and elongation to break according to the testing procedures set forth in the ASTM D 412-06a (2013) standard. This standard is available from ASTM International, and details the standard specifications and testing standards used for testing vulcanized rubber and Thermoplastic elastomers. These tests can be applied to multilayer films and gloves (such as examination and surgical gloves for medical applications).

### DETAILED DESCRIPTION

The elastomeric film-forming composition, dipped articles, gloves, methods of manufacture and uses thereof, according to particular embodiments of the invention are described below.

The present invention relates, in particular, to compositions containing (a) a carboxylated butadiene-based elastomer, (b) polychlorobutadiene in an amount of less than 30% by weight of the polymer content of the composition, and (c) one or more cross-linking agents. The present invention also relates to dipped articles, such as gloves or other products, which are made from the composition. It will be appreciated that the composition of the invention could be modified, such as by the addition of additives or by altering the relative amounts of other components, to suit the purpose of the dipped article or glove made from the composition.

Elastomeric Film-Forming Composition

The elastomeric film-forming composition comprises a dispersion or emulsion of a blend of polymer components (a) and (b) in a liquid. The composition generally comprises the polymers as well as cross-linking agents (c) in the liquid medium.

The liquid medium is typically water, although other solvents such as alcohols (including aliphatic alcohols and aromatic alcohols) or aromatic solvents may be used. Preferably, the solvent used is water. When water is used, the polymer is in colloidal form and processing and handling are simplified.

The total solids content of the elastomeric film-forming composition is from 5% to 45% by weight of the composition. The percentage of total solids content (TSC %) can vary within this range. Preferably, the total solids content of the elastomeric film-forming composition is about 5 to 42%, 10 to 45%, 10 to 42%, 15% to 45%, 15% to 42%, 20% to 45%, 20% to 42%, 5% to 40%, 10% to 40%, 20% to 40%, 30% to 45%, 30% to 42%, 30% to 40%, 35% to 45%, 35% to 40%, 5% to 35%, 7% to 35%, 8% to 35%, 9% to 35%, 10% to 35%, 11% to 35%, 12% to 35%, 13% to 35%, 20% to 35%, 30% to 35%, 5% to 30%, 7% to 30%, 8% to 30%, 9% to 30%, 10% to 30%, 11% to 30%, 12% to 30%, 13% to 30%, 20% to 30%, 5% to 28%, 7% to 28%, 8% to 28%, 9% to 28%, 10% to 28%, 11% to 28%, 12% to 28%, 13% to 28%, 20% to 28%, 5% to 25%, 7% to 25%, 8% to 25%, 9% to 25%, 10% to 25%, 11% to 25%, 12% to 25%, 13% to 25%, or 20% to 25%.

Generally, for forming a thin or disposable type of glove such as a surgical glove or medical examination type glove, the total solids content will be towards the lower end of this range. For example, the total solids content may be within one of the following ranges: 5 to 40%, 10 to 40%, 5 to 38%, 10 to 38%, 5 to 35%, 7% to 35%, 8% to 35%, 9% to 35%, 10 to 35%, 11% to 35%, 12% to 35%, 13% to 35%, 15% to 35%, 17% to 35%, 20% to 35%, 30% to 35%, 5% to 30%, 7% to 30%, 8% to 30%, 9% to 30%, 10 to 30%, 11% to 30%, 12% to 30%, 13% to 30%, 15% to 30%, 17% to 30%, 20% to 30%, 5% to 25%, 10 to 25%, 5% to 20%, 7% to 20%, 8% to 20%, 9% to 20%, 10 to 20%, 15% to 40%, 15% to 38%, 15% to 35%, 15% to 30%, 15% to 25%, 15% to 20%, 20% to 45%, 20% to 40%, 20% to 35%, 20% to 30%, 20% to 25%, 25% to 35%, 35% to 40% or 35-45%. For forming thicker gloves such as household gloves or industrial gloves, the total solids content will tend to be higher or the glove will be produced from many more layers. Thus, for thicker gloves, the total solids content will tend to be within one of the following ranges: 15 to 45%, 20 to 45%, 25 to 45%, 30% to 45%, 35% to 45%, 40-45%, 5 to 42%, 10 to 42%, 15 to 42%, 20 to 42%, 25 to 42%, 30% to 42%, 35% to 42%, 5% to 40%, 10 to 40%, 15 to 40%, 20 to 40%, 25 to 40%, 30% to 40% or 35% to 40%.

The elastomeric film-forming composition of the invention can be used to form a self-supported or unsupported film. A self-supported or unsupported film is a film that exists without other structural components or layers that the film is adhered to or attached to.

In the art of the present invention, it is common to refer to the amount of the polymer as being 100 phr (per hundred parts “rubber”), and for the relative amounts of the remaining components of the elastomeric film-forming composition to be calculated as a number of parts compared to the 100 phr of the polymer, by weight. Thus, for an amount of cross-linking agent that is 1/100th that of the polymer in the composition by weight, the amount of cross-linking agent is referred to as 1.0 phr.

It is also common in the art to use the expression “latex” or “rubber” to refer to any polymer in a general sense. Accordingly, particularly in the examples which follow, it should be understood that these terms have been used as short-hand to refer to the polymer of the dipping composition.

Component (a) Carboxylated Butadiene-Based Elastomer

The term “carboxylated butadiene-based elastomer” refers to any butadiene-based elastomer, which has been carboxylated.

Butadiene-based elastomers are homopolymers of butadiene (CH₂═CH—CH═CH₂) and copolymers of butadiene with one or more other monomers. Carboxylated butadiene-based elastomers are those containing some butadiene segments, rather than being all based on substituted butadiene. For example, polychloroprene, in which the butadiene contains a chlorine substitution at the 2-position, is not a “butadiene-based elastomer” in the context of component (a). It will be understood that the butadiene is not a substituted butadiene, other than by way of carboxylation, if this is achieved through carboxylate substitution of the units derived from butadiene (—CH₂—CH═CH—CH₂—) in the butadiene-based elastomer.

The butadiene-based elastomer may be based on a copolymer of butadiene with one or more other monomers. The other monomers (also referred to as “additional monomers”) may be selected from the group consisting of vinyl monomers such as acrylonitrile, styrene (vinyl benzene), vinyl acrylate, and butadiene derivative such as alkyl substituted butadiene, such as isoprene, which contains a single methyl group substitution at the 2-position of butadiene. In the case of the use of a butadiene derivative as the, or one of the, other monomers included in a copolymer with butadiene, there may be one or more substituents on the butadiene derivative, and these may be the same or different.

In preferred embodiments, each other monomer in the copolymer of butadiene with one or more other monomers is selected from the group consisting of vinyl monomers. In some embodiments, each other monomer is selected from the group consisting of acrylonitrile, vinyl benzene, or a combination thereof. In some embodiments, the carboxylated butadiene-based elastomer is carboxylated nitrile butadiene rubber, or carboxylated styrene-butadiene rubber, or carboxylated acrylonitrile-styrene-butadiene rubber. In some embodiments, the carboxylated butadiene-based elastomer is carboxylated nitrile butadiene rubber.

Carboxylation refers to the addition of, or inclusion, of a carboxylate group (—CO₂—), in the polymer. Carbon/late groups include carboxylic acid groups and ester groups. Carboxylation may be performed by way of grafting of carboxylic acid residues or esters thereof onto the polymer chain, or by way of copolymerising a carboxylic acid or ester-group containing monomer with the butadiene monomer (and any other monomers) in the production of the butadiene-based elastomer. Examples of suitable carboxylic acid-containing monomers include methacrylic acid, acrylic acid, crotonic acid, fumaric acid, maleic acid, citraconic acid, glutaconic acid, or terepthalic acid. Examples of suitable carboxylic acid ester-containing monomers are vinyl acetate, methyl acrylate, methacrylate ester, ethylenediol dimethacrylate, butanediol dimethacrylate (for example, the commercially available 1,3, BDDMA by BASF could be used), methyl methacrylate (for example, the commercially available MMA by The DOW Chemical Company or Rohm&Haas), butyl methacrylate (BMA) and glacial methacrylic acid (GMAA), other related acrylate monomers or combinations thereof.

Techniques for grafting or copolymerisation to achieve carboxylation of the butadiene-based elastomer are well known in the art. In addition, carboxylated butadiene-based elastomers are readily available from a wide range of elastomer suppliers in the field of the invention. A wide range of commercially available carboxylated butadiene-based elastomers can be used. These include commercially available carboxylated nitrile butadiene rubber, carboxylated styrene butadiene rubber, and other forms of carboxylated butadiene-copolymer rubbers.

Where components (a) and (b) are the only polymer components in the composition, the amount of component (a) may be from just above 70% to just under 100% of the total polymer content of the composition. The amount in this case may be between 71% to 99%, 71%-95%, 71-90%, 71-85%, 71-80%, 75-99%, 75-95%, 75-90%, 75-85%, 75-80%, 80-99%, 80-95%, 80-90%, 85-99%, 85-95% or 85-90%, of the total polymer content of the composition.

The relative amounts of (a):(b) may between 71:29 and 99:1 or any other ratio therebetween based on the percentages indicated above for (a)—such as 75:25 to 95:5 (corresponding to the % of 75%-95% indicated above). These ratios apply irrespective of whether the composition contains a further elastomer component.

Where there is a further elastomer component in the composition, the amount of component (a) may be 70% or lower, and may extend to as low as 30% by weight of the polymer content of the composition. In some embodiments, the amount of component (a) is greater than 40% by weight of the polymer content of the composition. The amount may be from 30-99%, 40-99%, 42-99%, 45-99%, 50-99%, 60-99%, 30-95%, 40-95%, 42-95%, 45-95%, 50-95%, 60-95%, 30-90%, 40-90%, 42-90%, 45-90%, 50-90%, 60-90% or otherwise.

Component (b) Polychiorobutadiene

The polychiorobutadiene is a non-carboxylated polychiorobutadiene.

Polychlorobutadiene refers to a butadiene-based polymer containing one or more chlorine substituents in the butadiene unit. The polychlorobutadiene may be a homopolymer based on one type of chlorobutadiene monomer, or a copolymer based on two or more different chlorobutadiene monomers. In some embodiments, the polychlorobutadiene is a homopolymer.

The polychlorobutadiene may be selected from polychloroprene (2-chlorobuta-1,3-diene), 2,3-dichlorobuta-1,3-diene, 1-chlorobuta-1,3-diene, 1,2-dichlorobuta-1,3-diene, 1,3-dichlorobuta-1,3-diene and 1,4-dichlorobuta-1,3-diene, by way of example.

In some embodiments, the polychlorobutadiene is polychloroprene. In some embodiments, the polychlorobutadiene is a copolymer of 2-chlorobuta-1,3-diene and 2,3-dichlorobuta-1,3-diene.

The relative amounts of different chlorobutadiene monomers used to produce the polychlorobutadiene will affect the overall amount of chlorine in the polychlorobutadiene component (i.e. component (a) of the composition). In order to produce a polychlorobutadiene having a specific level of chlorination, the polychlorobutadiene can be prepared by adjusting the relative amounts of chlorobutadiene and dichlorobutadiene used to form the polychlorobutadiene. In order to produce a copolymer having a specific level of chlorination, the copolymer can be prepared by adjusting the relative amounts of chlorobutadiene and dichlorobutadiene used to form the copolymer.

In one embodiment, the polychlorobutadiene comprises from about 10 to about 60% chlorine by weight of the chlorobutadiene units present in the polymer. Preferably, the polymer comprises from about 10% to about 58%, about 25% to about 60%, about 25% to about 58%, about 30% to about 60%, about 30% to about 58%, about 30% to about 45% or about 35% to about 45% chlorine by weight of the chlorobutadiene units present in the polymer. More preferably, the polymer comprises about 40% chlorine by weight of the total polymer.

Where the chlorine content is at the lower end of this range, the resulting dipped article will be softer, more stable and of nominal strength. Where the chlorine content is at the higher end of this range, the resulting dipped article will be tougher.

The stability of polychloroprene in general is poor compared to other latexes due to decomposition by autocatalytic dehydrochlorination. Preferably the pH of the elastomeric film-forming composition containing the polychlorobutadiene is maintained in the range of from about 8.5 to about 13.5 during formulation of the film-forming composition and production of the elastomeric articles. Preferably, the polymer has a pH in the range of from about 8.5 to 11, 9.0-11.5, 9.5-12, 10-12.5, 11-13, 11.5-13.5. It will be appreciated that the pH could be modified, such as by the addition of acid or base to suit the purpose of the composition.

The amount of component (b) in the composition is less than 30% of the total polymer content of the composition. The amount may be above 0% and less than 30% of the total polymer content of the composition. The amount may be between 1-29%, 5-29%, 10-29%, 1-27%, 5-27%, 10-27%, 1-25%, 5-25%, 10-25%, 15-29%, 15-27%, 15-25%, 15-20%, 1-20%, 5-20%, 10-20%, 15-20% or otherwise.

Further Elastomers

In some embodiments, the carboxylated butadiene-based elastomer (component (a)) and polychlorobutadiene (component (b)) are the only elastomers present in the composition.

In other embodiments, a further elastomer may be included in the blend. Examples of suitable further elastomers include synthetic elastomers or synthetic rubbers such as nitrile rubber, styrene butadiene rubber, butyl rubber, polyisoprene, polyvinylchloride, polyurethane, styrene diblock copolymers, styrene triblock copolymers, acrylic polymers or other synthetic elastomers or mixtures thereof. The further elastomer may be carboxylated (for example, by grafting or copolymerizing and or mixtures thereof), non-carboxylated, or a mixture of carboxylated and non-carboxylated elastomers, or a mixture of elastomers having varied degrees of carboxylation.

The amount of the further elastomer used will depend on the polymer that is used and the end product to be produced.

Preferably the amount of any third (or further) elastomer will be less than 50% of the total polymer content of the composition, and in some embodiments it is less than 40%, or less than 30% or less than 20%, or less than 10% of the composition. In some embodiments, the amount of any third (or further) elastomer will be greater than 10% of the total polymer content of the composition, and in some embodiments it is greater than 12%, or greater than 15%. It will be appreciated that any of the upper and lower limits on the amount of any third (or further) elastomer can be combined to provide a range for the amount of any third (or further) elastomer included in the composition. It will be appreciated that the presence of the further elastomer cannot be so high as to adversely impact on the favourable properties provided by the use of components (a) and (b) recited above.

Cross-Linking Agents

The cross-linking agent or agents present in the composition serve to cross-link the polymers to produce an elastomeric film. One or more cross-linking agents of various types can be used. Cross-linking agent classes include ionic cross-linking agents and covalent cross-linking agents. The cross-linking agent or agents used in the production of the elastomeric film may be selected from ionic cross-linking agents, covalent cross-linking agents, and combinations thereof. The selection will depend on various factors including the properties of the film desired and the choice of elastomer.

Accelerators are one sub-class of cross-linking agents which release sulphur, or act with sulphur-containing compounds, to accelerate sulphur-based covalent cross-linking of the elastomer-forming polymer. Generally, accelerators can be advantageous as they shorten the curing (vulcanisation) time, lower the curing temperature or decrease the amount of cross-linking agents required to be used in the composition. However, on the negative side, accelerators can give rise to allergic reactions, such as allergic contact dermatitis with symptoms including erythema, vesicles, papules, pruritus, blisters and/or crusting. Examples of suitable accelerators include the carbamates such as thiocarbamates (e.g. zinc dibutyl dithiocarbamate (ZDBC), Zinc diethyl dithiocarbamate (ZDEC)); thiurams (e.g. tetraethylthiuram disulfide (TETD), Tetramethylthiuram disulphide (TMTD) and dipentamethylenethiuram tetrasulfide (DPTT)); thiourea (Ethyl thiourea (ETU) and diphenylthiourea (DPTU); thiazoles (e.g. Mercapto Benzothiazoles (MBT), Mercapto Benzothiozole disulphide (MBTS), zinc 2-mercaptobenzothiazole (ZMBT)); guanidines (e.g. Diphenylguanidine (DPG)) and aldehyde/amine-based accelerators (e.g. hexamethylenetetramine). Other examples are well known in the art and can be obtained from various publicly available sources.

Another class of cross-linking agents are the ionic cross-linking agents, which include metal oxides, metal hydroxides and peroxides (organic and inorganic). These work by ionically cross-linking ionically-crosslinkable groups in the elastomer-forming polymer. For example, a metal oxide cross-linker can work by ionically cross-linking the carboxylic acid groups of the polymer comprising chlorobutadiene units and one or more carboxylic acid residues or esters thereof. Examples of suitable metal oxide cross-linking agents include the multivalent metal oxide cross-linking agents, such as lead oxide, magnesium oxide, barium oxide, zinc oxide, manganese oxide, copper oxide, aluminium oxide, nickel oxide, and combinations thereof. Example of a suitable metal hydroxide cross-linking agents include zinc hydroxide, aluminium hydroxide, magnesium hydroxide, and other metal hydroxides, such as barium hydroxide, manganese hydroxide, copper hydroxide and nickel hydroxide. An example of a peroxide cross-linking agent is 1,1-di(t-butylperoxy)-3,3,5-trimethylcyclohexane, which can be purchased under the trade name Trigonox 29-40B-pd. Other cross-linking agents that are suitable for use in the elastomeric film-forming composition are selected from, but not restricted to cross-linking monomers, reactive oligomers, polyisocyanate oligomers, functional, cross-linkable polymers, derivatives of ethylene glycol di(meth)acrylate (such as ethylene glycol diacrylate, di(ethylene glycol) diacrylate, tetra(methylene/ethylene glycol) diacrylate, ethylene glycol dimethacrylate (EDMA), di(ethylene glycol) dimethacrylate (DEDMA), tri(methylene/ethylene glycol) dimethacrylate, tetraethylene glycol dimethacrylate (TEDMA)), derivatives of methylenebisacrylamide (such as N,N.-methylenebisacrylamide, N,N-methylenebisacrylamide, N,N.-(1,2 dihydroxyethylene)bisacrylamide), formaldehyde-free cross-linking agents (such as N-(1-Hydroxy-2,2-dimethoxyethyl)acrylamide), divinylbenzene, divinylether, diallyl phthalate, divinylsulfone and the like. Some of these cross-linking agents are commercially available and are supplied by companies such as Aldrich. Combinations of these cross-linking agents can also be used.

The amount of cross-linking agent is typically in the range 0.1-15.0 phr. In some embodiments, the amount of cross-linking agent is suitably within one of the following ranges: 0.1-15.0 phr, 0.1-13.0 phr, 1.0-11.0 phr, 0.1-10.0 phr, 0.1-8.0 phr, 0.1-7.0 phr, 0.1-6.0 phr, 0.1-5.0 phr, 0.1-4.0 phr, 0.1-3.0 phr, 0.5-15.0 phr, 1.0-15.0 phr, 1.5-15.0 phr, 0.5-13.0 phr, 1.0-13.0 phr, 1.5-13.0 phr, 0.5-11.0 phr, 1.0-11.0 phr, 1.5-11.0 phr, 0.5-10.0 phr, 1.0-10.0 phr, 1.5-10.0 phr, 0.5-8.0 phr, 1.0-8.0 phr, 1.5-8.0 phr, 0.5-7.0 phr, 1.0-7.0 phr, 1.5-7.0 phr, 2.0-8.0 phr, 2.5-10.0 phr, 5.0-10.0 phr, 3.0-7.0 phr, 0.5-6.0 phr, 1.0-6.0 phr, 2.0-6.0 phr, 3.0-6.0 phr, 4.0-7.0 phr, 4.0-6.0 phr, 4.0-5.0 phr, 2.0-5.0 phr, 2.0-4.0 phr, 3.0-4.0 phr, 6-10 phr, 7-10 phr, 6-8 phr, 5-9 phr, 8-10 phr, 0.1-3.5 phr, 0.1-2.0 phr, 0.1-1.5 phr, 0.1-1.0 phr or 0.1-0.5 phr.

A metal oxide can serves two functions in the elastomeric film-forming compositions of the present invention. Firstly the metal oxide can neutralize hydrochloric acid that is formed from the slow dehydrochlorination of the chlorobutadiene units, and secondly, the metal oxide can cross-link the functional groups to provide excellent bond strength and heat resistance. The allyl chloride structures in the polymer of component (a) and the carboxylic acid residues or esters thereof in component (b) function as major cross-linking sites by reaction with metal oxides. The amount of metal oxide that will typically be used is between 0.01 to 10 parts, or 0.01 to 2 parts, per hundred parts of dry rubber.

The suitable vulcanization activators comprise metal oxides, such as lead oxide, magnesium oxide, barium oxide, zinc oxide, manganese oxide, copper oxide, aluminium oxide and nickel oxide, preferably zinc oxide.

A further class of cross-linking agents are the covalent cross-linking agents, which include sulphur and sulphur-containing vulcanising agents. These work by covalently cross-linking unsaturated double bonds present in the elastomer-forming polymer. The sulphur can be present in the form of elemental sulphur. The sulphur in sulphur-containing vulcanising agents can also be donated by organic sulphuric compounds, for example TMTD (Tetramethylthiuram Disulfide). Sulphur donors or sulphur-containing vulcanising agents such as this one are likely to contribute to chemical allergies and it is preferred to keep their use to a minimum in the manufacture of gloves when allergic content is an issue. Thus, if used, the sulphur is preferably present in the form of elemental sulphur.

Generally, the amount of cross-linking determines the elasticity of the elastomeric film. Therefore, the amount and type of cross-linking agent will contribute to the extent of cross-linking and the elasticity of the final elastomeric film.

For ionic cross-linking agents such as metal oxide and peroxide cross-linking agents, when used, the amount is typically in the range 0.01-10.0 phr. The amount of metal oxide cross-linking agent is suitably within one of the following ranges: 0.01-10.0 phr, 0.5-10.0 phr, 1.0-10.0 phr, 1.5-10.0 phr, 2.5-10.0 phr, 5.0-10.0 phr, 6.0-10 phr, 7.0-10 phr, 8.0-10 phr, 5.0-9.0 phr, 0.01-8.0 phr, 0.5-8.0 phr, 1.0-8.0 phr, 1.5-8.0 phr, 2.0-8.0 phr, 6-8 phr, 0.5-7.0 phr, 1.0-7.0 phr, 1.5-7.0 phr, 3.0-7.0 phr, 4.0-7.0 phr, 3.0-6.0 phr, 4.0-6.0 phr, 4.0-5.0 phr, 2.0-5.0 phr, 0.01-5.0 phr, 2.0-4.0 phr, 3.0-4.0 phr, 0.01-3.5 phr, 0.01-3.0 phr, 0.01-2.0 phr, 0.01-1.5 phr, 0.01-1.0 phr, 0.02-1.0 phr, 0.05-1.0 phr, 0.1-1.0 phr, 0.2-1.0 phr, 0.25-1.0 phr, 0.01-0.75 phr, 0.02-0.75 phr, 0.05-0.75 phr, 0.1-0.75 phr, 0.2-0.75 phr, 0.25-0.75 phr or 0.01-0.5 phr. In some embodiments, the metal oxide is zinc oxide and is used in an amount of less than 2 phr, for example, within one of the following ranges: 0.01-1.8 phr, 0.01-1.5 phr, 0.01-1.0 phr, 0.02-1.0 phr, 0.05-1.0 phr, 0.1-1.0 phr, 0.2-1.0 phr, 0.25-1.0 phr, 0.01-0.75 phr, 0.02-0.75 phr, 0.05-0.75 phr, 0.1-0.75 phr, 0.2-0.75 phr, 0.25-0.75 phr or 0.01-0.5 phr.

Sulphur requires high energy at curing (thus high curing temperature and/or time) compared to other cross-linking agents. However, sulphur does provide the resulting dipped articles, such as gloves, with greater chemical resistance, and therefore it may be desired for this reason. The amount of sulphur is suitably within one of the following ranges: 0.0-3.5 phr, such as 0.01-3.5 phr, 0.01-3.0 phr, 0.01-2.0 phr, 0.01-1.5 phr, 0.01-1.0 phr, 0.01-0.5 phr, 0.1-3.5 phr, 0.1-3.0 phr, 0.1-2.0 phr, 0.1-1.5 phr, 0.3-1.5 phr, 0.5-3.5 phr, 0.5-3.0 phr, 0.5-2.0 phr, 0.5-1.5 phr, 0.5-1.0 phr, 0.6-3.5 phr, 0.6-3.0 phr, 0.6-2.0 phr, 0.6-1.5 phr, 0.6-1.0 phr, 0.7-3.5 phr, 0.7-3.0 phr, 0.7-2.0 phr, 0.7-1.5 phr, 0.7-1.0 phr, 0.8-3.5 phr, 0.8-3.0 phr, 0.8-2.0 phr, 0.8-1.5 phr or 0.8-1.0 phr.

In some embodiments, where the amount of carboxylic acid or ester in component (b) is higher, it could be possible to reduce and even eliminate accelerators from the elastomeric film-forming composition of the invention. For example, for dipped articles having a larger film thickness, accelerator elimination is feasible where the strength is not compromised. However, further improved physical characteristics may be obtained using an accelerator, such as further improved softness. Where this property is desirable, it will be preferable to use sufficient accelerators. Accordingly, the composition for producing the elastomeric film will be accelerator-free in some embodiments, and will further comprise an accelerator in other embodiments.

The amount of (total) accelerator is suitably between 0.1-3.0 phr, such as between 0.1-3.0 phr, 0.1-2.5 phr, 0.1-2.0 phr, 0.1-1.5 phr, 0.1-1.0 phr, 0.2-3.0 phr, 0.2-2.5 phr, 0.2-2.0 phr, 0.2-1.5 phr, 0.2-1.0 phr, 0.3-3.0 phr, 0.3-2.5 phr, 0.3-2.0 phr, 0.3-1.5 phr, 0.3-1.0 phr, 0.4-3.0 phr, 0.4-2.5 phr, 0.4-2.0 phr, 0.4-1.5 phr, 0.4-1.0 phr, 0.5-3.0 phr, 0.5-2.5 phr, 0.5-2.0 phr, 0.5-1.5 phr, or 0.5-1.0 phr. Suitable accelerators include mercaptobenzothiazoles and derivatives thereof, dithiocarbamates and derivatives thereof, sulphur donors, guanidines, thio-urea and aldehyde-amine reaction products.

In one embodiment, the cross-linking agents used in the elastomeric film-forming composition of the present invention are selected from the group consisting of sulphur, a sulphur-containing vulcanising agent, organic peroxide, metal oxide, metal hydroxide and combinations thereof. Preferably, the composition contains a combination of sulphur or a sulphur-containing vulcanising agent, and a metal oxide or metal hydroxide. The use of the combination of cross-linking agents, such as sulphur and metal oxide, provides a polymer having ionic cross-linking as well as covalent cross-linking across the unsaturated double bonds of the polymer. The metal oxide will form ionic bonds to the carboxylic acid or ester groups and to the chlorine. Formation of ionic bonds requires less energy and allows quicker production of the elastomeric film-forming composition. The sulphur will form covalent bonds with the butadiene, particularly at carbon sites. Formation of these covalent bonds requires higher energy, however, the resulting elastomeric film may have improved permeation characteristics. Accordingly, the combination of these types of cross-linking agents provides a balance between the time and energy required to produce the elastomeric film and the performance of the elastomeric film. The combination of ionic and covalent cross-linking, in the copolymer may also provided an elastomeric film having improved properties, such as improved strength and durability of the film. The amount and type of cross-linking also contributes to the elasticity of the film.

Other Components or Additives

Other components or additives may be included in the composition can include one or more additives selected from the group consisting of plasticizers, antiozonants, stabilisers such as pH stabilisers, emulsifiers, antioxidants, vulcanising agents, polymerisation initiators, pigments, fillers, colourising agents and sensitisers.

Stabilisers may be used in the elastomeric film-forming composition. The stabilizer may be, for example, an oleate, stearate or other non-ionic surfactants. The elastomer-forming polymer can be diluted with a solution of a stabilizer, such as potassium hydroxide, ammonium hydroxide and/or sodium hydroxide. The amount of stabiliser used is dependent on the polymer used in the elastomeric film-forming composition, the pH of the composition and other factors. The stabiliser can range from 0.1-5.0 phr, e.g. 0.5 to 2 phr, preferably 1.0 to 1.5 phr, which is diluted with water, preferably filtered water or de-ionized water, or water having a total solid content of around 5 ppm level-water.

Emulsifiers may be used in the elastomeric film-forming composition. Suitable emulsifiers include sodium alkyl sulphates or other non-ionic and ionic surfactants. The amount of emulsifier used is dependent on the on the polymer used in the elastomeric film-forming composition, the pH of the composition and other factors. The amount of emulsifier can range from about 0.1 to 5 phr, 0.5 to 5 phr, 0.1 to 3 phr or 0.5 to 3 phr.

pH stabilisers may be used to avoid the possibility of destabilization, which is possible where the elastomer-forming polymer contains carboxylic acid groups. Suitable pH stabilisers include potassium hydroxide, ammonium hydroxide and/or sodium hydroxide. Preferably, the pH stabiliser is potassium hydroxide. A diluted stabilizer solution can be mixed with the elastomer-forming polymer. The pH of the mixture is suitably adjusted to between about 8.5 to about 13.5, or between about 8.5 to about 11.0. The cross-linking agent(s) can then be added to the mixture. The amount of pH stabilizer can range from about 0.1 to 3.0 phr, 0.1 to 2.5 phr, 0.1 to 2.0 phr, 0.1 to 1.5 phr, 0.1 to 1.0 phr, 0.1 to 0.5 phr, 0.2 to 3.0 phr, 0.2 to 2.5 phr, 0.2 to 2.0 phr, 0.2 to 1.5 phr, 0.2 to 1.0 phr, 0.2 to 0.5 phr, 0.5 to 3.0 phr, 0.5 to 2.5 phr, 0.5 to 2.0 phr, 0.5 to 1.5 phr or 0.5 to 1.0 phr.

Antiozonants may be used in the elastomeric film-forming composition. Suitable anitozonants include paraffinic waxes, microcrystalline waxes and intermediate types (which are blends of both paraffinic and microcrystalline waxes). The amount of antiozonant can range from about 0.1 to 5.0 phr, 0.1 to 3.0 phr, 0.1 to 1.5 phr, 0.5 to 5.0 phr, 0.5 to 3.0 phr, or 0.5 to 1.5 phr.

Antioxidants may be added to the elastomeric film-forming composition of the present invention. Suitable antioxidants include hindered arylamines or polymeric hindered phenols, and Wingstal L (the product of p-cresol and dicyclopentadiene). The antioxidant may, for example, be added in an amount ranging from about 0.1-5.0 phr, such as about 0.1-3.0 phr, 0.5-3.0 phr, 0.1-1.5 phr, 0.1-1.0 phr or 0.3-0.5 phr.

Pigments, such as titanium dioxide, are selected for their pigmentation, or to reduce the transparency of the final elastomeric film. Pigments may also be referred to as opaqueness providers. The amount of pigment may, for example, be added in amounts ranging from about 0.01-10.0 phr, such as 0.01-5.0 phr, 0.01-3.0 phr, 0.01-2.0 phr, 0.01-1.5 phr, or 1.5-2.0 phr and colorants can also be added in the desired amounts. The mixture is then diluted to the target total solids concentration by the addition of a liquid, such as water. The pigments used in the elastomeric film-forming composition may be selected from the group consisting of EN/USFDA approved dyes.

Rubber reoderants may be used in the elastomeric film-forming composition. Suitable rubber reoderants include perfume oils of natural or synthetic origins. The amount of rubber reoderant can range from about 0.001 to 2.0 phr.

Wetting agents may be used in the elastomeric film-forming composition. Suitable wetting agent emulsifiers include anionic surfactants like sodium dodecyl benzene sulphonate or sodium lauryl ether sulphate, or non-ionic ethoxylated alkyl phenols such as octylphenoxy polyethoxy ethanol or other non-ionic wetting agents. The amount of wetting agent can range from about 0.001 to 2.0 phr.

Defoamers or anti-foam may be used in the elastomeric film-forming composition. Defoamers may be chosen from naphthalene type defoamers, silicone type defoamers and other non-hydrocarbon type defoamers or defoamers of refined vegetable origin. The amount of defoamers can range from about 0.001 to 2.0 phr, such as about 0.001-1.0 phr, 0.001-0.1 phr, 0.001-0.01 phr.

The elastomeric film-forming composition may also contain an inorganic filler. Suitable inorganic fillers include calcium carbonate, carbon black or clay. Preferably, the amount of inorganic filler included in the blend would not exceed 30% either alone or in combination. It will be appreciated that the blended composition will retain the favourable properties provided by the use of components (a) and (b).

Sensitisers are chemicals that can be used in compositions for producing elastomeric films to control the amount of the composition that will remain coated on the mould during dipping. Examples of sensitisers known in the art that can be used in the composition for producing an elastomeric film include polyvinyl methylether, polypropylene glycol, ammonium nitrate and ammonium chloride. When used, the amount of sensitiser will be chosen based on the desired film thickness to remain on the mould during dipping, and will generally be between 0.01-5.0 phr. For thinner films, the amount will generally be between about 0.01 to 2.0 phr, such as about 0.1 to 1.0 phr. When other techniques are used for controlling the film thickness on the mould, such as the use of pre-dipping the mould into coagulant before undertaking the multiple dipping into the composition for producing the elastomeric film, the composition for producing an elastomeric film may not comprise a sensitiser.

Those skilled in the art will readily be able to vary the components of the elastomeric film-forming composition to suit the particular polymer used as well as the particular final article desired. It will also be understood by those of skill in the art that specific chemicals or compounds which have been listed above are intended to be representative of conventional materials that may be used in formulating the elastomeric film-forming composition and are merely intended as non-limiting examples of each such component of the composition.

Preparation of the Elastomeric Film-Forming Composition

The composition for producing an elastomeric film can be prepared by mixing components (a), (b) and (c), and any of the optional further components, in a liquid (e.g. water). The process may involve pre-preparation of single components at a particular concentration (total solids content), diluting those components if desired, combining, and undertaking any further dilution as required to reach the final total solids content set for the composition.

Suitable additives or other components as described above may be included in the composition, and may be added to a combination of components (a) and (b) before addition of the cross-linking agent (c), or added to the mixture of all of components (a), (b) and (c).

Typically, the powder components of the composition will be combined and milled using suitable milling equipment to reduce the particle size to a suitable range. Preferably, the average particle size is below 5 microns. Uniform particle size is desirable, and coarse milling may result in non-uniform particles and therefore a non-uniform film, which can result in high fluctuation in film properties.

When used, the surfactant and the pH stabilizer are added to the liquid (e.g. water) and mixed properly without any foam formation. This liquid is then used to dilute the elastomer components ((a) and (b)), and other additives or components to the desired total solids content. The total solids content of the elastomeric film-forming composition will depend on the planned film thickness.

The pH of the dispersion may then be adjusted as necessary, preferably to a pH within the range of 8.5 to 13.5 (e.g. a pH above 9 or preferably a pH between 10 and 11). Any high variation between the diluted polymer and dispersion will result in coagulation from the micro level to the macro level.

When the components have been mixed uniformly or to homogeneity, other additives such as colorants and emulsifiers are added. The elastomeric film-forming composition is then left for maturation. The length of the maturation may vary depending on the level of cross-linking agent and the degree of carboxylation of the polymer. Generally, the composition will be left for a minimum of 12 to 18 hours, while in some cases maturation could be conducted over a period of days depending upon the requirements for preparing the dipped article and the level of cross-linking agents present. The compounded elastomeric film composition with suitable additives could be prematured by holding the composition at a controlled elevated temperature. For example, the elastomeric film composition could be held at 20° C. to 60° C. for a period of, for example, about 4 hours to about 24 hours depending on the temperature, degree of carboxylation of the polymer, the amount and type of vulcanization activators and accelerators, and type and quantity of pH stabilizer and emulsifier stabilizer and wetting agents/surfactants.

Preparation of the Elastomeric Film

The elastomeric film-forming composition having the desired composition is formed into the shape of the desired article, and then dried and/or cured. Curing is used in a general sense, to refer to the stage during which cross-linking is performed. Such curing conditions are as known in the art.

Any known techniques can be used to form the desired shape of elastomeric article, including dipping processes, extrusion and otherwise. Dipping processes are preferred. This may be performed on conventional equipment known in the art.

Set out below are brief details of one suitable technique for producing an elastomeric article using a dipping process. This is described in the context of producing a thin film glove. It should be understood that variations may be made to this process as known or described in the art. The steps in the manufacture of a film may be as generally described in PCT/AU2014/000726 and PCT/AU2014/000727, which are incorporated by reference.

Optional Step (a) Dipping the Former into a Coagulant Containing Multivalent Ions in Solution

The details of this step are as described in the PCT publications referred to above. In brief, a suitable former, which is based on the shape of the article to be produced (e.g. flat for a film or glove-shaped for a glove) can be dipped into a coagulant containing multivalent ions in solution. The former is dipped into a coagulant containing multivalent ions, leaving a thin coating of the charged ions on the surface of the former. The charged ions coating can assist in controlling the amount composition for forming the elastomeric film that will subsequently remain on the surface of the mould after dipping into the composition, through charge interactions. The composition of the coagulant may be as described in the two PCT publications as described above. Cationic multivalent ion-containing coagulates are typically used, such as a calcium coagulant.

Optional Step (b) Drying or Partially Drying the Coagulant-Dipped Former

If the former is dipped into a coagulant, following this step the former is dried or partially dried.

Step (i) Dipping the Former into the Elastomeric Article-Forming Composition of the Invention to Produce a Layer of Elastomeric Article-Forming Composition on the Mould

The former is dipped into the elastomeric film-forming composition, embodiments of which have been described in detail above. The duration of dipping, temperature, and former surface temperature may be as described in the PCT publications referred to above.

Step (ii) Drying or Partially Drying the Layer of Elastomeric Film-Forming Composition on the Former

The conditions and details of this step may be as described in the PCT publications referred to above.

The method of manufacture described herein encompasses the preparation of single-layered or multiple-layered elastomeric films. Therefore, in some embodiments, the method may include step (v), which involves drying and curing the layered elastomeric film on the former directly after this step to prepare a single layered elastomeric film. In other embodiments, the method may include a number of repetitions of optional steps (iii) and (iv) after this step to produce a multiple-layered elastomeric film.

Step (iii) Optionally Dipping the Former Coated with the Dried or Partially Dried Layer of Elastomeric Film-Forming Composition into the Elastomeric Film-Forming Composition to Produce a Further Layer of Elastomeric Film-Forming Composition on the Former

This step is optional, and is present when multi-layer articles are produced. The details of this step are as described in the PCT publications referred to above.

Step (iv) Optionally Repeating the Drying or Partial Drying Step (ii) and the Further Dipping Step (iii)

This step is optional, and is present when multi-layered articles are produced. The number of layers may be 2, 3 or more in multi-layered articles. The details of this step are as described in the PCT publications referred to above.

Step (v) Optional Additional Steps Prior to Drying and/or Curing

Further steps can be taken to fine-tune the manufacture of the elastomeric film or article. The details of these steps are as described in the PCT publications referred to above. In brief, the film or article can be leached to remove extractable components, there may be a coating material applied, beading/cuffing cab be performed and/or the product may be passed through a curing or vulcanizing oven to evaporate the water in the film and enable better cross linking.

Step (vi) Drying and/or Curing the Layered Elastomeric Film on the Former

The details of this step are as described in the PCT publications referred to above.

Step (vii) Additional Steps

In any suitable sequence, addition optional steps that can be performed prior to stripping of the glove from the former include cooling, chlorination, post-curing rinsing, polymer coating and additional drying steps. The cured film may also be cooled/chlorinated/neutralized—post-leached in hot water and optionally dipped in lubricant solution or any silicone/silicone free polymers to enable easy stripping and better donning.

Step (viii) Stripping

The film or article is stripped from the former at the conclusion of the formation process.

Dipped Articles and Use of the Elastomeric Film-Forming Composition

The elastomeric film-forming composition of the present invention can be used to prepare a variety of dipped articles. Examples of possible dipped articles include surgical gloves and medical examination gloves, industrial gloves, finger cots, catheters, tubing, protective coverings, balloons for catheters, condoms and the like. Preferably, the elastomeric film-forming composition is used in the manufacture of gloves, such as powder-free gloves.

The thickness of the final film (or article) can, for example, be in the range 0.01-3.0 mm, such as 0.01-2.5 mm, 0.01-2.0 mm, 0.01-1.5 mm, 0.01-1.0 mm, 0.01-0.5 mm, 0.01-0.4 mm, 0.01-0.3 mm, 0.01-0.2 mm, 0.01-0.15 mm, 0.02-2.5 mm, 0.02-2.0 mm, 0.02-1.5 mm, 0.02-1.0 mm, 0.02-0.5 mm, 0.02-0.4 mm, 0.02-0.3 mm, 0.02-0.2 mm, 0.01-0.10 mm, 0.02-0.15 mm, 0.02-0.1 mm, 0.03-3.0 mm, 0.03-2.5 mm, 0.03-2.0 mm, 0.03-1.5 mm, 0.03-1.0 mm, 0.03-0.5 mm, 0.03-0.4 mm, 0.03-0.3 mm, 0.03-0.2 mm, 0.03-0.15 mm, 0.03-0.10 mm, 0.05-3.0 mm, 0.05-2.5 mm, 0.05-2.0 mm, 0.05-1.5 mm, 0.05-1.0 mm, 0.05-0.5 mm, 0.05-0.4 mm, 0.05-0.3 mm, 0.05-0.2 mm, 0.05-0.15 mm, 0.05-0.10 mm, 0.08-3.0 mm, 0.08-2.5 mm, 0.08-2.0 mm, 0.08-1.5 mm, 0.08-1.0 mm, 0.08-0.5 mm, 0.08-0.4 mm, 0.08-0.3 mm, 0.08-0.2 mm, 0.08-0.15 mm, 0.08-0.10 mm, 0.1-3.0 mm, 0.1-2.5 mm, 0.1-2.0 mm, 0.1-1.5 mm, 0.1-1.0 mm, 0.1-0.5 mm, 0.1-0.4 mm, 0.1-0.3 mm, 0.1-0.2 mm, 0.15-3.0 mm, 0.15-2.5 mm, 0.15-2.0 mm, 0.15-1.5 mm, 0.15-1.0 mm, 0.15-0.5 mm, 0.15-0.4 mm, 0.15-0.3 mm, 0.15-0.2 mm, 0.02-0.08 mm, 0.03-0.08 mm, or 0.05-0.08 mm. In some embodiments, the thickness of the final film (or article) can, for example, be in the range 0.01-0.10 mm or 0.05-0.08 mm for thin or disposable gloves, and in the range 0.1-3.0 mm for thick gloves.

In some embodiments, thick films are made of multiple thin layers of film to reach the desired thickness.

The thickness is suitably measured as an “average thickness”, particularly for gloves, using the points of measurement described below. In some embodiments, the film thickness of a glove is less than 2 mm (e.g. from 0.01 mm to 2 mm). For example, the film thickness may be in the range of from 0.04 mm to 2 mm.

In another embodiment, the glove may have a weight of about 4 g, while it will be appreciated that higher and lower glove weights may also be obtained depending on the purpose for which the glove is to be used.

The final film (or article) can, for example, have one layer or be made from multiple layers produced by separate dipping steps. For example, the final film (or article) may comprise from 1 to 15 layers.

The dipped articles prepared from the elastomeric film-forming composition of the invention also possess improved physical properties. In some embodiments, the dipped articles prepared from the elastomeric film-forming composition of the invention have a higher tensile strength, a lower modulus at 300% and/or a lower modulus at 500% and a higher elongation to break when compared to other elastomeric to form a dipped articles or gloves. In some embodiments, the dipped articles prepared from the elastomeric film-forming composition of the invention have a lower modulus at 300%, a lower modulus at 500% and/or a higher elongation to break when compared to other elastomeric films used to form dipped articles or gloves. In some embodiments, the dipped articles prepared from the elastomeric film-forming composition of the invention have a tensile strength of greater than or equal to about 2000 psi, a modulus at 300% of about 100 to 2000 psi, a stress at 500% of about 200 to 3000 psi, and/or an elongation to break of about 400 to 1500%. For example, the elastomeric film prepared from the composition of the present invention has a modulus at 300% of less than about 650 psi, a stress at 500% no greater than about 1500 psi, and/or an elongation to break of greater than 550%. For example, the elastomeric film prepared from the composition of the present invention has a tensile strength of at least about 2000 psi, a modulus at 300% of less than about 650 psi, a stress at 500% no greater than about 1500 psi, and/or an elongation to break of greater than about 550%. In some embodiments, the elastomeric film prepared from the composition of the present invention has a tensile strength of 2000 psi to 4000 psi. In some embodiments, the elastomeric film prepared from the composition of the present invention has a modulus at 300% of 200 psi to 650 psi. In some embodiments, the elastomeric film prepared from the composition of the present invention has a stress at 500% of 200 psi to 1500 psi. In some embodiments, the elastomeric film prepared from the composition of the present invention has an elongation to break of greater than 600%. Preferably, the elastomeric film prepared from the composition of the present invention has an elongation to break of 550% to 1100%.

The elastomeric film-forming composition of the invention can be used to form elastomeric films or dipped articles in which the softness of the film ranges from very soft to medium to very rigid by varying the amounts of the components used in the composition and the type of components used in the composition. In some embodiments, the softness of the elastomeric film or dipped article can be varied by adjusting the level of carboxylation of the polymer/copolymer, the amount and type of the second elastomer used in the composition, the amount and type of cross-linking agent or agents, and/or the amount of chlorine in the polymer/copolymer. As one example, the elastomeric film prepared from the composition of the present invention may be used to form a soft film having a tensile strength of greater than or equal to about 2100 psi, a modulus at 300% of less than or equal to about 660 psi, a stress at 500% of less than or equal to about 1015 psi, and/or an elongation to break of greater than about 800%. As another example, the elastomeric film prepared from the composition of the present invention may be used to form a soft to medium film having a tensile strength of greater than or equal to about 2100 psi, a modulus at 300% of less than or equal to about 1200 psi, a stress at 500% of less than or equal to about 2800 psi, and/or an elongation to break of about 500 to 800%. As a further example, the elastomeric film prepared from the composition of the present invention may be used to form a medium to rigid film having a tensile strength of greater than or equal to about 2100 psi, a modulus at 300% of less than about 1200 psi, a stress at 500% of less than about 2800 psi, and/or an elongation to break of about 400 to 700%.

The desired durability of the film is determined by the end use of the article. For example, for gloves for non-surgical use, the wearing time is usually below 3 hrs, and commonly less than 2 hrs. The durability of the film can be controlled by the curing conditions. Generally, the higher the curing temperature, the more durable the elastomeric film.

The term “average thickness” in respect of the thickness of a glove (specifically the multi-layer elastomeric film forming the glove) refers to the average of three thickness measurements, taken at points along the layer of the elastomeric film. The measurements are taken at the cuff, the palm and the finger tip. When measuring the thickness of individual layers of the glove, the “average thickness” is a reference to the average thickness of that layer of film, taken at the three measurement points. This may be measured in absolute terms (in mm), or as a percentage of the full thickness of the multi-layered glove. For elastomeric articles, a similar technique using three thickness measurements can be used to determine the “average thickness”.

In the claims and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

The invention is illustrated by the following examples.

### EXAMPLES

The invention will now be described in further detail with reference to the following non-limiting examples. All testing procedures are shown in the Testing Procedures section, and the results of these tests are shown. All tables of compositions and test results are shown in the Tables section.

General Procedure

In the examples set out below, the following general procedure was utilised to produce elastomeric films, and gloves in particular. The general procedure was also used to demonstrate the impact (if any) that certain processing conditions and components of the elastomeric film forming compositions have on the quality of multilayer elastomeric films produced.

The following general procedure was followed for the all the Examples (1-5) described below.

1. Washing

The formers are subjected to pre-washing, so as to be clean of any remaining residues following removal of a glove previously made on the former. The formers are cleaned in mild acid/alkali and hot water. The formers are then dried by blowing air by blowers or air curtains or using ovens with the hot air having temperature above 105° C.

2. Coagulant Dipping

The cleaned dry former is immersed in the coagulant bath, which contains a 0-50% by weight solution of calcium nitrate. The coagulant also contains 0.1%-5.0% by weight metallic stearates, suitable wetting agents (0.001-1.0%) and antifoaming agents (0.001-1.0%).

3. Drying

The coagulant coated formers are dried in a hot air circulated oven at a temperature of about 110° C. to 130° C.

4. First Dipping Step

The former, coated with dried coagulant, is dipped into a tank of the composition for forming an elastomeric film, which contains the components specified for the given example. The composition used has a concentration of about 5 to 60% by weight, and preferably 10-40% by weight. The composition is maintained at temperature of around 20-35° C., and is constantly circulated in the tank to avoid creaming and settling of chemicals. The former is dipped into the composition for a dwell time of 5 seconds to 60 seconds.

5. Drying

The composition coated formers are gelled in a gelling oven at a temperature of about 100-300° C. and the duration of 2-300 seconds.

6. Pre-Leaching

Pre-leaching is conducted by rinsing in warm water for a short period of time. The gelled film coating on the former is pre-leached in series of tanks at ambient temperature to 55° C.

7. Optional Second Dipping Step

Then pre-leached gelled film coating on the former is optionally dipped into a tank of the composition for forming an elastomeric film, which contains the components specified for the given example. If performed, the composition has a concentration of about 5 to 50%, and preferably 8-35% by weight. The composition is maintained at temperature of around 10-60° C., and preferably 20-40° C., and is constantly circulated in the tank to avoid creaming and settling of chemicals. The former is dipped into the composition for a dwell time of 5-90 seconds. The optional second dipping step was not performed for Examples 1 to 5 of this application.

8. Gelling/Pre Leaching/Beading

The product is subjected to gelling and pre-leaching and beading.

The beading, drying and pre-leaching steps could be carried out in any order. The processes of beading and pre-cure leaching could be exchange depending on the quality of cuff beading.

9. Vulcanization

The beaded glove is then vulcanized at about 100° C.-150° C. for about 15-30 minutes depending upon the film thickness.

10. Post-Leaching/Lubricant/Final Drying/Stripping/Tumbling

The vulcanized glove will be post leached and lubricant dipped (optional) and stripped after final drying. Where additional curing or surface treatment is required, the gloves could be tumbled using hot air at a temperature around 80-120° C. for about 15-120 minutes.

General Formulation

The generic glove formulation is as follows:

TABLE 1

| | Parts per Hundred Rubber |
|---|---|
| Ingredients | (phr) - Dry basis |
| Carboxylated nitrile butadiene rubber* | Up to 99% of elastomer |
| | content |
| Polychlorobutadiene (non carboxylated) | <30% |
| Third optional elastomer | 0-50% of elastomer |
| | content |
| TOTAL ELASTOMER AMOUNT | 100 phr |
| Plasticizer stabilizer | 0.5-5.0, when present |
| Emulsifier stabilizers | 0.5-5.0, when present |
| Antiozonant | 0.5-5.0, when present |
| pH stabilizer | 0.1-3.0, when present |
| Ionic crosslinking agent | 0.01-8.0, when present |
| Cross-linker | 0.01-3.0 in preferred |
| | embodiments |
| Vulcanisation accelerators | 0.1-4.0, when present |
| Antioxidant | 0-3.0, when present |
| Opaqueness provider | 0.01-3.0, when present |
| Pigment | As per requirement |
| Defoamer | 0.001-2.0 |

*Commercially available carboxylated nitrile butadiene rubber. Suppliers of suitable carboxylated butadiene-based elastomers include Synthomer, Nippon Zeon, Khumho, LG and NanTex.

The pH stabilizers may be for example oleates, stearates or other non-ionic surfactants or potassium hydroxide, ammonium hydroxide and or sodium hydroxide.
The emulsifier stabilizers may be sodium alkyl sulphates, potassium salts of resin/rosin acids or other non-ionic surfactants.
The antiozonants may be paraffinic waxes, microcrystalline waxes and intermediate types.
Ionic crosslinking agents are multivalent metal oxides.
The cross-linker may be sulphur and/or other organic peroxides and/or cross linkable reactive monomers.
The vulcanization accelerator is chosen from mercaptobenzothiazoles and derivatives, dithiocarbamates and derivatives, sulphur donors, guanidines and its derivatives, thiourea and its derivatives and aldehyde amine reaction products.
The antioxidant may be hindered polymeric phenols or arylamines. Opaqueness provider could be titanium oxide or other minerals.
Defoamer may be naphthalene type defoamers, vegetable oil based defoamers, silicone type defoamers and like.

Carboxylated Butadiene-Based Elastomer (Component (a))

The carboxylated butadiene-based elastomer may be purchased from a supplier such as Synthomer Sdn Bhd.

Polychloroprene (Component (b))

The component (b) is non-carboxylated polychloroprene. The non-carboxylated polychloroprene used in the examples had a medium to high gel content and a pH above 12.0. Such non-carboxylated polychoroprenes are available from a range of suppliers including Denka and Showa Denko, Japan.

### Examples 1 to 9

Formulations having the compositions indicated in Tables 2 and 3 are based on different relative amounts of polychloroprene to carboxylated butadiene-based elastomer. In Examples 1 to 5, there was a variation in the amount of cross-linking agents and other components present in the compositions. Examples 6 to 9 were then prepared, based on fixed amounts of the cross-linking agents and other components (based on the amounts used in Example 5), and variations only in the relative amount of polychloroprene to carboxylated butadiene-based elastomer. When the results of Examples 6 to 9 and 5 are taken together, they show the trend when varying the relative amount of elastomers from 5 to 27%.

Gloves are prepared from the composition following the General Procedure indicated above.

TABLE 2

| | Ex. 1 | Ex. 2 | Ex. 3 | Ex. 4 | Ex. 5 |
|---|---|---|---|---|---|
| Polychloroprene | 5 | 10 | 15 | 20 | 27 |
| ZnO | 0.25 | 0.35 | 0.45 | 0.55 | 0.75 |
| ZDBC | 0.15 | 0.2 | 0.3 | 0.4 | 0.5 |
| DPTU | 0.15 | 0.2 | 0.3 | 0.4 | 0.5 |
| ANTIOXIDANT | 0.5 | 0.6 | 0.7 | 0.8 | 1 |
| TIO2 | 2 | 2 | 2 | 2 | 2 |
| Carboxylated NBR | 95 | 90 | 85 | 80 | 73 |
| KOH | 1.7 | 1.7 | 1.5 | 1.5 | 1 |
| AGWET | 0.3 | 0.3 | 0.4 | 0.4 | 0.5 |
| SULPHUR | 0.5 | 0.6 | 0.7 | 0.8 | 1 |
| DPTT | 0.15 | 0.2 | 0.3 | 0.4 | 0.5 |

ZDBC, DPTU (diphenyl thiourea) and DPTT (dipentamethylenethiuram tetrasulfide) are accelerators.
ZnO is an ionic cross-linking agent.
Agwet is a surfactant (sodium dodecyl benzene sulfonate).

TABLE 3

| | Ex. 6 | Ex. 7 | Ex. 8 | Ex. 9 | Ex. 5 |
|---|---|---|---|---|---|
| Polychloroprene | 5 | 10 | 15 | 20 | 27 |
| ZnO | 0.75 | 0.75 | 0.75 | 0.75 | 0.75 |
| ZDBC | 0.5 | 0.5 | 0.5 | 0.5 | 0.5 |
| DPTU | 0.5 | 0.5 | 0.5 | 0.5 | 0.5 |
| ANTIOXIDANT | 1 | 1 | 1 | 1 | 1 |
| TIO2 | 2 | 2 | 2 | 2 | 2 |
| Carboxylated NBR | 95 | 90 | 85 | 80 | 73 |
| KOH | 1 | 1 | 1 | 1 | 1 |
| AGWET | 0.5 | 0.5 | 0.5 | 0.5 | 0.5 |
| SULPHUR | 1 | 1 | 1 | 1 | 1 |
| DPTT | 0.5 | 0.5 | 0.5 | 0.5 | 0.5 |

Abbreviations as in Table 2.

The films formed from the compositions prepared in accordance with Examples 1 to 9 were found to meet the requirements of current ASTM specifications for gloves made of synthetic elastomeric compositions. It will be appreciated that different properties or standards could be selected and the compositions adjusted as appropriate for the standards and/or customer requirements for a variety of fields of applications.

The films were uniform and no weak spot or pin holes were observed. The glove thickness varied from 0.05 to 0.10 from cuff end to the finger tip. The elongation was better than typical nitrile butadiene rubber products. The modulus was lower than that achieved with typical nitrile butadiene rubber products.

Test Procedures

The following testing techniques are used for testing the properties of the films produced.

General Testing Procedures

Tensile strength, stress at 300% and 500% modulus and elongation to break are measured by testing procedures conducted in accordance with ASTM D 412-06a (2013). This standard is available from ASTM International, and details the standard specifications and testing standards used for testing vulcanized rubber and thermoplastic elastomers. These tests can be applied to films and gloves (such as examination gloves for medical applications).

The tests are performed on unaged films (i.e. films as produced from the film compositions described above), and on aged films (i.e. films that have undergone an accelerated aging process to simulate the effect of aging of the film over an extended time period—typically three years.) The accelerated aging conditions are set out in ASTM D6319, and involve subjecting the film to a temperature of 100° C. for 22 hours.

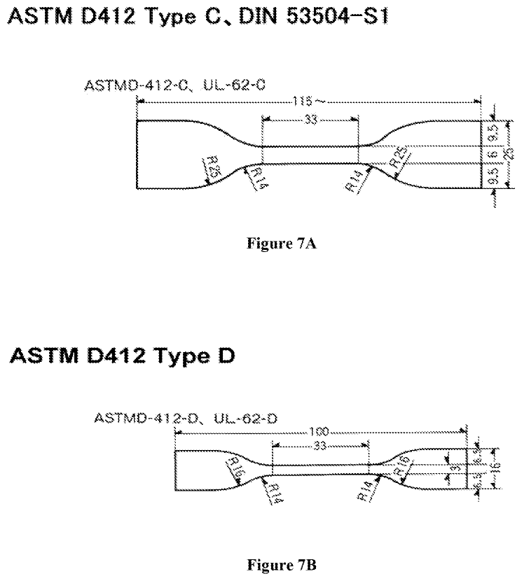

FIG. 7A is a schematic diagram showing an ASTM D412 Type C sample cutting die. FIG. 7B is a schematic diagram showing an ASTM D412 Type D sample cutting die. The dies are used to prepare test specimens for testing according to the ASTM D 412-06a standard.

Results

The elastomeric films prepared using the elastomeric film-forming compositions of Examples 1 to 9 were tested, and the following properties of the elastomeric films were measured for both the unaged (“BA”) and aged (“AA”) films:
Modulus at 300%
Modulus at 500%
Tensile strength (MPa); and
Elongation %.

The results are set out in Tables 4 to 7 below. The results are grouped in two collections.

TABLE 4

| | | UNAGED | | | | |
|---|---|---|---|---|---|---|
| Expt. | % | Tensile | Mod@100 | Mod@ 300 | Mod@500 | Elongation |
| No. | CR | (Mpa) | (Mpa) | (Mpa) | (Mpa) | (%) |
| Ex. 1 | 5 | 21.6 | 0.97 | 1.57 | 2.87 | 790 |
| Ex. 2 | 10 | 20.57 | 1.11 | 1.86 | 3.75 | 730 |
| Ex. 3 | 15 | 23.24 | 1.05 | 1.85 | 3.88 | 730 |
| Ex. 4 | 20 | 25.43 | 1.26 | 2.27 | 5.23 | 700 |
| Ex. 5 | 27 | 21.67 | 1.17 | 2.32 | 6.42 | 650 |

TABLE 5

| | | AGED | | | | |
|---|---|---|---|---|---|---|
| Expt. | % | Tensile | Mod@100 | Mod@ 300 | Mod@500 | Elongation |
| No. | CR | (Mpa) | (Mpa) | (Mpa) | (Mpa) | (%) |
| Ex. 1 | 5 | 30.93 | 1.24 | 2.43 | 5.81 | 690 |
| Ex. 2 | 10 | 32.34 | 1.25 | 2.62 | 6.92 | 670 |
| Ex. 3 | 15 | 30.55 | 1.47 | 3.17 | 10.21 | 620 |
| Ex. 4 | 20 | 39.71 | 1.86 | 4.28 | 14.39 | 620 |
| Ex. 5 | 27 | 32.12 | 1.93 | 5.26 | 23.41 | 550 |

TABLE 6

| | | UNAGED | | | | |
|---|---|---|---|---|---|---|
| Expt. | % | Tensile | Mod@100 | Mod@ 300 | Mod@500 | Elongation |
| No. | CR | (Mpa) | (Mpa) | (Mpa) | (Mpa) | (%) |
| Ex. 6 | 5 | 21.94 | 0.9 | 1.52 | 3.33 | 780 |
| Ex. 7 | 10 | 18.57 | 0.81 | 1.49 | 3.15 | 760 |
| Ex. 8 | 15 | 20.79 | 0.97 | 1.77 | 3.86 | 730 |
| Ex. 9 | 20 | 22.2 | 0.97 | 1.75 | 3.75 | 760 |
| Ex. 5 | 27 | 21.67 | 1.17 | 2.32 | 6.42 | 650 |

TABLE 7

| | | AGED | | | | |
|---|---|---|---|---|---|---|
| Expt. | % | Tensile | Mod@100 | Mod@ 300 | Mod@500 | Elongation |
| No. | CR | (Mpa) | (Mpa) | (Mpa) | (Mpa) | (%) |
| Ex. 6 | 5 | 41.46 | 1.39 | 3.22 | 9.12 | 650 |
| Ex. 7 | 10 | 34.19 | 1.35 | 2.91 | 8.24 | 640 |
| Ex. 8 | 15 | 35.63 | 1.35 | 3.1 | 9.01 | 630 |
| Ex. 9 | 20 | 35.1 | 1.32 | 2.82 | 7.98 | 650 |
| Ex. 5 | 27 | 32.12 | 1.93 | 5.26 | 23.41 | 550 |

Analysis

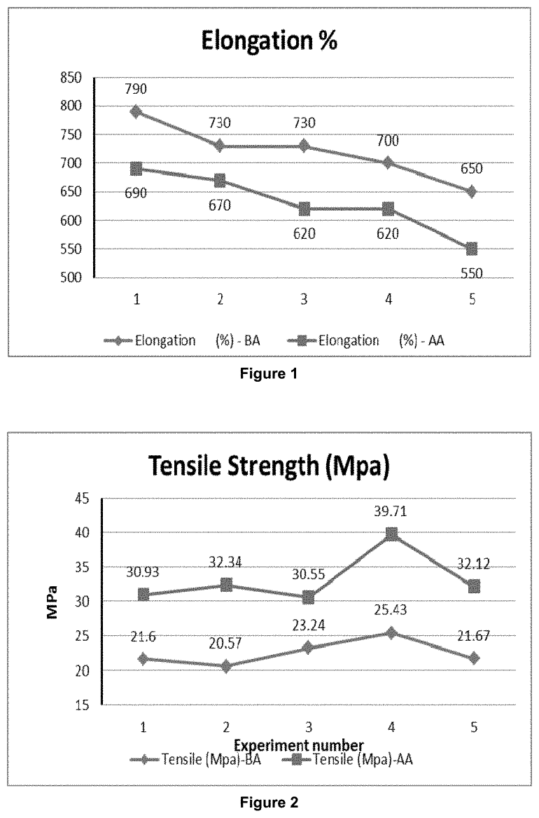

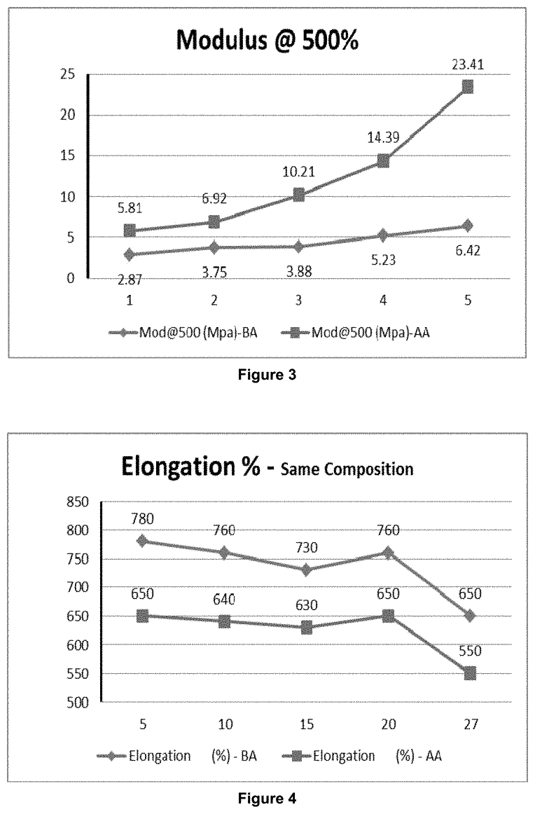

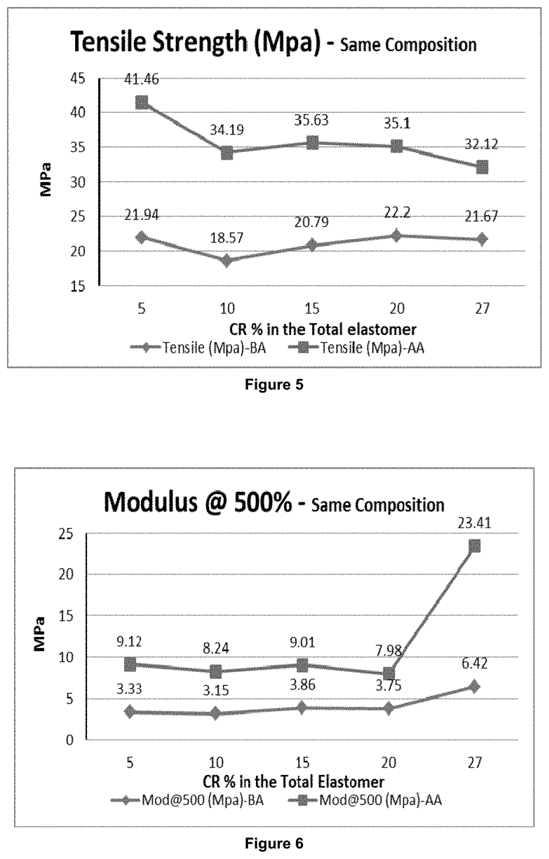

The film testing results have been graphed, and the graphs appear in FIGS. 1, 2 and 3 for Examples 1 to 5, and FIGS. 4, 5 and 6 for Examples 6 to 9 and 5, to show the trends produced when increasing the amount of polychloroprene in the blend.

On review of the results and figures, the following points are noted:
For optimal products, it is desired to achieve a balance between a high tensile strength and low modulus (which represents softness), particularly following an accelerated aging process.
The results obtained for the first set of Examples (1 to 5) show that tensile strength, particularly under accelerated aging conditions, peaked at about 20% of polychloroprene content, and dropped off after that. The tensile strength results obtained for the consistent formulations (with increasing polychloroprene content—Examples 6 to 9 and 5) showed good values for after-aging results across the range, with highest tensile strength value coming from the gloves with 5% polychloroprene content. This correlates to the highest relative amount of carboxylated nitrile butadiene rubber. The results for the first set of Examples (1 to 5) also show that the modulus at 500% for products containing up to 20% polychloroprene remains acceptably low, particularly following aging, with the slope of the graph up to the Example 3 point remaining reasonably low, and only increasing slightly from the Example 3 point to the Example 4 point. The rate of increase in the modulus from 27% and above indicates a rapid increase in the modulus, indicating significantly lower softness is expected from around 30% polychloroprene content and above. Very similar results are obtained across Examples 6 to 9 and 5, with the modulus only increasing markedly (especially after aging) when moving from 20% polychloroprene to 27% polychloroprene. The trend in elongation shows high elongation %, with the elongation after aging only dropping to 550 (and following this trend, lower) once the polychloroprene content increases above 27%.
The combination of these results supports a range of polychloroprene content being maintained in the region of about 1% polychloroprene to just under 30% polychloroprene content—for example, around 5% polychloroprene to 27% polychloroprene, or 5% to 25% polychloroprene, or 5% to 20% polychloroprene, or 5% to 15 polychloroprene or 5% to 10% polychloroprene

The foregoing description and examples relate only to preferred embodiments of the present invention and numerous changes and modifications may be made therein without departing from the spirit and scope of the invention as defined in the following claims.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-36. (canceled)

37. An elastomeric film-forming composition comprising:
(a) a carboxylated butadiene-based elastomer,
(b) polychlorobutadiene in an amount of less than 30% by weight of the polymer content of the composition,
(c) cross-linking agents including 0.01-0.5 phr of ionic cross-linking agent, 0.01-1.0 phr sulphur and 0.1-1.0 phr of one or more accelerators, and a total cross-linking agent amount of 0.1-2.5 phr, and
(d) a total solids content of 5% to 30% by weight of the composition,
wherein all of component (b) is non-carboxylated polychlorobutadiene.

38. The elastomeric film-forming composition of claim 37, wherein component (a) is selected from the group consisting of carboxylated nitrile butadiene rubber, carboxylated styrene-butadiene rubber and carboxylated acrylonitrile-styrene-butadiene rubber.

39. The elastomeric film-forming composition of claim 37, wherein the amount of component (a) is between 71% to 99% by weight of the total polymer content of the composition.

40. The elastomeric film-forming composition of claim 37, wherein the composition comprises a further elastomer, and the amount of component (a) is between 30-99% by weight of the total polymer content of the composition.

41. The elastomeric film-forming composition of claim 37, wherein the amount of component (b) in the composition is between 5-25% by weight of the total polymer content of the composition.

42. The elastomeric film-forming composition of claim 37, comprising a further elastomer in an amount of less than 50% by weight of the total polymer content of the composition.

43. The elastomeric film-forming composition of claim 37, wherein components (a) and (b) are the only polymer components of the composition.

44. The elastomeric film-forming composition of claim 37, wherein the amount of the ionic cross-linking agent is 0.35 phr or less.

45. An elastomeric article comprising at least one layer of a cured composition comprising:
(a) a carboxylated butadiene-based elastomer,
(b) polychlorobutadiene in an amount of less than 30% by weight of the polymer content of the composition, and
(c) cross-linking agents including 0.01-0.5 phr of ionic cross-linking agent, 0.01-1.0 phr sulphur and 0.1-1.0 phr of one or more accelerators, and a total cross-linking agent amount of 0.1-2.5 phr,
wherein the elastomeric article has a thickness of 0.01-0.10 mm and all of component (b) is non-carboxylated polychlorobutadiene.

46. The elastomeric article of claim 45, wherein component (a) is carboxylated nitrile butadiene rubber, carboxylated styrene-butadiene rubber, carboxylated acrylonitrile-styrene-butadiene rubber.

47. The elastomeric article of claim 45, wherein the amount of component (a) is between 71% to 99% by weight of the total polymer content of the composition.

48. The elastomeric article of claim 45, comprising a further elastomer in an amount of less than 50% by weight of the total polymer content of the composition.

49. The elastomeric article of claim 45, wherein components (a) and (b) are the only polymer components of the composition.

50. The elastomeric article of claim 45, wherein the amount of ionic cross-linking agent is 0.35 phr or less.

51. The elastomeric article of claim 45, in the form of a glove or condom.

52. The elastomeric article of claim 45, in the form of a glove having an average thickness based on the average of the cuff, palm and finger thicknesses of between 0.01 mm and 0.10 mm.

53. The elastomeric article of claim 45, with a tensile strength of greater than or equal to about 2100 psi (14.5 MPa) and an elongation to break of about 500 to 800%.

54. The elastomeric article of claim 45, comprising from 2 to 15 elastomeric film layers.

55. A method of manufacturing an elastomeric glove comprising the steps of:
(i) preparing the elastomeric film-forming composition of claim 37, the preparation of the elastomeric film-forming composition including the steps of:
mixing a diluted pH stabiliser with the carboxylated butadiene-based elastomer to produce a diluted polymer; followed by
combining the diluted polymer with other components of the composition without any high variation in the pH so as to avoid coagulation, to produce the elastomeric film-forming composition,
(ii) dipping a former into said elastomeric film-forming composition to produce a layer of elastomeric film-forming composition on the former,
(iii) drying and/or curing the elastomeric film-forming composition, and
(iv) stripping the product of step (iii) from the former to produce an elastomeric glove with a thickness of 0.01-0.1 mm.

56. An elastomeric glove produced from the composition of claim 37.
